(19) United States
(12) Patent Application Publication
(10) Pub. No.: US 2025/0058144 A1
(43) Pub. Date: Feb. 20, 2025

(54) NEGATIVE-PRESSURE DRUG LOADING APPARATUS AND NEGATIVE-PRESSURE DRUG LOADING METHOD

(71) Applicant: SUZHOU YIBEN LIFE TECHNOLOGY CO., LTD, Suzhou (CN)

(72) Inventors: Guoxiang SUN, Suzhou (CN); Yonghua DONG, Solon, OH (US); Liangliang ZHAO, Suzhou (CN); Lei JIN, Suzhou (CN)

(73) Assignee: SUZHOU YIBEN LIFE TECHNOLOGY CO., LTD, Suzhou, JS (CN)

(21) Appl. No.: 18/822,443

(22) Filed: Sep. 2, 2024

Related U.S. Application Data
(63) Continuation-in-part of application No. PCT/CN2023/079157, filed on Mar. 1, 2023.

(30) Foreign Application Priority Data
Mar. 1, 2022 (CN) 202210197666.5

(51) Int. Cl. A61N 5/10

(52) U.S. Cl. CPC A61N 5/1007; A61N 2005/1009; A61N 2005/1024; A61N 2005/1092

(57) ABSTRACT

Disclosed are a negative-pressure drug loading apparatus and a negative-pressure drug loading method. When the apparatus is in use, drug particles to be loaded are placed into the particle storage tube, and a proper amount of drug solution is introduced. The particle storage tube is placed in the body, and the sealing space is sealed. A vacuum pump is used to execute a negative pressure operation in the body until the inside of the particle storage tube is pumped into a vacuum. An air valve of the vacuum pump is opened, such that the drug solution comes into the said particles under the action of pressure. After the pressure is stable, the particle storage tube is let stand for a period of time such that the drug solution stably stays in the said particles.

### BACKGROUND

### Technical Field

The disclosure relates to a negative-pressure drug loading apparatus and a corresponding negative-pressure drug loading method, belonging to a field of medical devices.

### Related Art

Implantation of drug loaded particles has become one of the commonly used means for the treatment of cancer. Drug loaded particles, such as radioactive ones, are created by encapsulating radionuclides within metal shells to destroy tumor cells. Despite their potential, the high production costs of these existing drug-loaded particles hinder their widespread adoption and practical application. Furthermore, drug loaded particles can also encompass nano-sized or micro-sized particles, such as chitosan-based drug loaded nanoparticles.

However, these micro-particles are not suitable for implantation procedures.

### SUMMARY

The primary technical problem to be solved by the present disclosure is to provide a negative-pressure drug loading apparatus to improve the convenience and efficiency of drug loading operation on radioactive particles.

Another technical problem to be solved by the disclosure is to provide a negative-pressure drug loading method.

In order to achieve the above purposes, the following technical solutions are used by the present disclosure.

According to a first aspect of an embodiment of the present disclosure, provided is a negative-pressure drug loading apparatus, for loading under negative-pressure unloaded particles placed in a particle storage tube, comprising:
a body having at least one mounting slot for positioning the particle storage tube, and
a cover configured to cover the body to provide a closed space under negative-pressure, and enable drug solution enters inner cavities of the unloaded particles through micro-holes opened on the unloaded particles.

Preferably, the mounting slot includes a guide cavity and an accommodation cavity,
the guide cavity is located above the accommodation cavity, and an inner diameter of the guide cavity is larger than that of the accommodation cavity.

Preferably, an inner diameter of the accommodation cavity is 0.6 mm˜1.2 mm to adapt to different specifications of the particle storage tubes.

Preferably, the cover is provided with a same number of inlets as the mounting slot, and the inlets are connected with the closed space for filling the drug solution into the particle storage tube or feeding the unloaded particles.

Preferably, the cover is also provided with a connection part for connecting with a vacuum pump, and the connection part is connected with the closed space.

Preferably, the body comprises a mounting bracket, an inner sleeve and an outer sleeve;
the mounting bracket is provided with a plurality of the mounting slots for installing the particle storage tubes;
the mounting bracket is placed in the inner sleeve;
the inner sleeve is placed in the outer sleeve;
the cover is configured to seal or reveal the outer sleeve, thereby forming the closed space;
the outer sleeve is suitable to connect with a vacuum pump.

Preferably, further comprising a housing, a control unit and a vacuum pump;
wherein the body and the vacuum pump are both arranged in the housing, and the cover can be opened and closed on the housing to seal or open the outer sleeve;
the control part is arranged in the housing to electrically connect with the vacuum pump and control the vacuum pump to pressurize or depressurize the closed space.

According to a second aspect of an embodiment of the present disclosure, provided is a negative-pressure drug loading method comprising the following steps:
placing a particle storage tube into a mounting slot and closing the cover to form a sealed space within the body;
utilizing a vacuum pump to decompress the sealed space until no bubbles appear in the particle storage tube;
stopping the vacuum pump and allowing the pressure in the sealed space to gradually become greater than or equal to the ambient pressure;
opening the cover and taking out the particle storage tube after the pressure stabilizes to the ambient pressure, wherein
the particle storage tube is initially empty, and before the decompression of the sealed space, the following steps are also included: through the inlet, sequentially placing a predetermined number of drug-loaded particles and a predetermined amount of drug solution into the particle storage tube; or, the particle storage tube already contains drug-loaded particles, and before the decompression of the sealed space, the following step is also included: through the inlet, injecting a predetermined amount of drug solution into the particle storage tube; or, the particle storage tube already contains both drug-loaded particles and a predetermined amount of drug solution.

Preferably, the predetermined amount of drug solution is determined according to the number and specification of the unloaded particles in the particle storage tube.

Preferably, during the operation of depressurization, the pressure and pumping speed of the vacuum pump are pre-set according to the amount of the drug solution, the volume in the housing portion, and the micro-holes' diameter, to ensure a balance where the pressure is high enough to allow the air inside the unloaded particles to be discharged but not so high that the drug solution is sucked out.

Compared with the prior art, the negative-pressure drug loading apparatus and the method provided by the disclosure have the following technical effects:
1) under the negative-pressure, unloaded particles with micro-holes less than 1 mm can be loaded with drug solutions, which improves the drug loading rate and loading ratio;
2) the operation is simple and efficient, enhancing the drug loading efficiency and convenience, requiring only 1-2 minutes to load drug solutions to the unloaded particles at a time.
3) the structure is portable and compact, suitable for use on doctors' desktops and/or for large-scale production.
4) it is low-cost and facilitated, making it suitable for widespread applications.

### BRIEF DESCRIPTION OF THE DRAWINGS

FIG. 1 is the structural diagram of a particle storage tube used in the present disclosure;

FIG. 2 is the structural diagram of a negative-pressure drug loading apparatus provided by the first embodiment of the disclosure;

FIG. 3 is a schematic diagram of the state of the particle storage tube after negative-pressure operation;

FIG. 4 is a schematic diagram of the state of the particle storage tube after pressurization;

FIG. 5 is a schematic diagram of the state of the particle storage tube after it has been standing for a preset length of time;

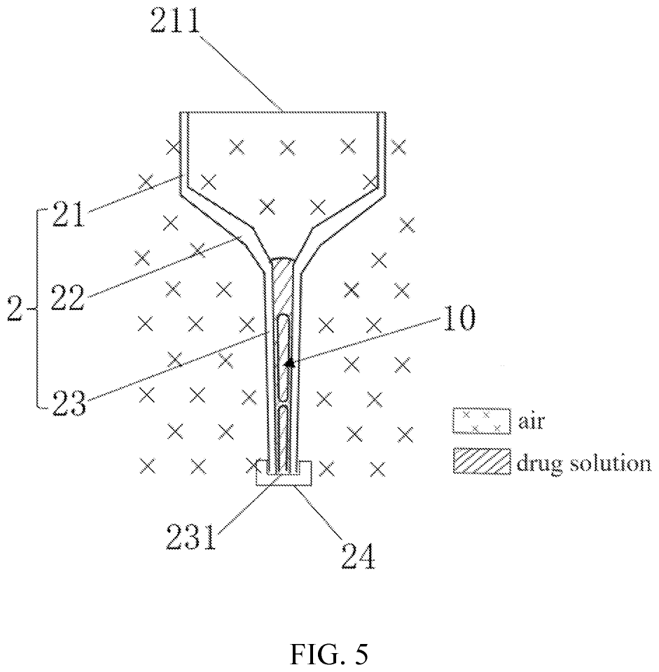

FIG. 6 is the structural diagram of a negative-pressure drug loading apparatus provided by the third embodiment of the present disclosure;

FIG. 7 is the structural diagram of a negative-pressure drug loading apparatus provided by the fourth embodiment of the present disclosure.

### DETAILED DESCRIPTION

The technical content of the present disclosure will be described in detail below in combination with the accompanying drawings and specific embodiments.

The drug loaded particle for implantation in the embodiment of the present disclosure refers to a particle whose size is suitable for implantation operation (0.1-5 mm in diameter, such as a cylindrical I-125 radioactive particle with a diameter of 0.8 mm and a length of 4.5 mm) and which contains liquid active ingredients within the particle. Particles here refer to microspheres, microcapsules or microparticles, and are not limited to specific shapes. The drug loaded particle has a shell, and a micro-hole (pore size less than or equal to 1 mm) is opened on the shell to allow the drug solution to enter the inner cavity from the outside of the shell. Before implantation operation, the unloaded particles need to be loaded with drugs in advance to prepare the drug loaded particles.

The negative-pressure drug loading apparatus provided by the embodiment of the disclosure is suitable for micro drug loading, that is, the drug solution loaded at one time is less than or equal to 100 microliters, which improves the drug loading capacity and the efficiency of drug loading operation.

### First Embodiment

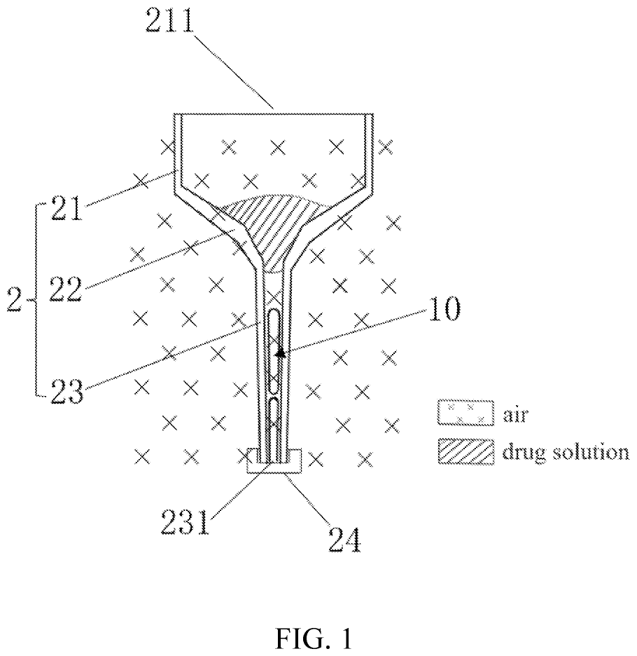

FIG. 1 shows the structural diagram of a particle storage tube used in the present disclosure. The particle storage tube 2 includes a guide portion 21, a transition portion 22, and a housing portion 23 that are sequentially communicated. One end of the guide portion 21 is used to match with a plug (not shown) to seal the guide portion 21; and one end of the housing portion 23 is used to match with a cap to seal. Therefore, in combination with the plug and the cap, the particle storage tube 2 can provide a cavity to seal and contain particles inside.

Specifically, the guide portion 21 is located at an open end of the particle storage tube 2. The shape of the guide portion 21 can be selected according to varied applications, for example, it can be a circular tube or a square tube. The guide portion 21 is provided with a first opening 211 to perform the following functions: 1) before loading drug, feeding an unloaded particle (a particle to be loaded with drugs) into the particle storage tube 2; 2) after feeding the unloaded particle, injecting the drug solution into the particle storage tube 2; 3) after completing drug loading, a plug (not shown) is accommodated to seal the particle storage tube 2.

The guide portion 21 has a first inner diameter, which is a preset size, usually several times the outer diameter of the unloaded particles, to achieve: 1) it should not be too small to avoid inconvenience in the automatic operation of inputting multiple particles; 2) it should not be too large to avoid inconvenience in storage and transportation. The first inner diameter is comprehensively set according to the pressure value of the negative-pressure equipment, the outer diameter of the unloaded particles, the pore size of the particles, and other factors

The housing portion 23 is located at an outlet end of the particle storage tube 2 and is used for discharging the drug-loaded particles during the implantation operation. The housing portion 23 features a second opening 231, which serves to release the drug-loaded particles that have been filled with the drug solution from the particle storage tube 2. Prior to drug loading, a cap 24 is plugged into the second opening 231 to seal it, allowing the particle storage tube 2 to be subjected to negative-pressure for hydraulic injection of the drug into the unloaded particle 10. Once the drug has been loaded under negative-pressure, the cap 24 can be removed, enabling the loaded particles to be taken out through the second opening 231.

The housing portion 23 features a second inner diameter that is smaller than the first inner diameter. This second inner diameter is of a preset size, selected based on the outer diameter of the unloaded particles 10. It is designed to be slightly larger than the outer diameter of the drug-loaded particles, ensuring that the small drug-loaded particles can be not only arranged in a row within the housing portion 23, but also slide smoothly within it. This design helps to prevent multiple particles from becoming stuck in the housing portion 23. The second inner diameter is of mm level, for example, 0.5 mm˜2 mm, which is suitable for drug loaded particles of different specifications. More specifically, the second inner diameter is 1 mm, and the drug is loaded into particles with an outer diameter of 0.8 mm. The unloaded particles are provided with micro-holes that allow the drug solution to enter for drug loading, or to exude into the human body after implantation. The pore is obtained by laser processing, and the preferred size is between 0.01 and 0.1 mm, less than 1 mm. The pore size is related to the release speed of the drug solution, and is pre-determined based on specific conditions. In this regard, a detailed description is provided in the applicant's previous application “MICROPARTICLE FOR DRUG LOADING, DRUG LOADING MICROPARTICLE, PARTICLE CONTAINING TUBE, AND IMPLANTATION SYSTEM FOR MICROPARTICLE” (CN111437265A, US 2023/0028772 A1). At the same time, the size of the second inner diameter should match the outer diameter of the unloaded particle 10, so as to facilitate the placement of the unloaded particle 10.

The transition portion 22 is connected between the guide portion 21 and the housing portion 23, and the inner diameter of the transition portion 22 is gradually reduced from the first inner diameter to the second inner diameter. Specifically, the guide portion 21, the transition portion 22, and the housing portion 23 form a funnel-shaped structure together. Those skilled in the art can understand that the transition part 22 can alternatively be omitted, that is, the guide portion 21 is directly connected with the housing portion 23.

In the above embodiment, the particle storage tube 2 can have a variety of specifications, and the second inner diameters of the particle storage tubes 2 of various specifications are different (preferably, they have the same first inner diameter) for matching with a variety of unloaded particles 10 with different outer diameters. Therefore, according to the different types of the unloaded particles 10, the particle storage tube 2 with appropriate specifications can be selected for drug loading, so as to ensure that the negative-pressure drug loading apparatus can adapt to the common types of particles in the markets.

In the described embodiment, the particle storage tube 2 is made of a transparent material and features a scale, which includes both scale lines and numerical values to indicate the volume of liquid medicine filled. Both the inner and outer sleeves are also made of transparent material, allowing the operator to clearly see the scale on the particle storage tube 2. Consequently, based on the number of unloaded particles 10 within the tube, the required volume of drug solution can be pre-calculated. This enables precise control of drug loading to meet the specific requirements. As an alternative, the automatic injection of drug solution into the particle storage tube can be adopted. In this case, there is no need for the scale value, and there is no need for a transparent material accumulator.

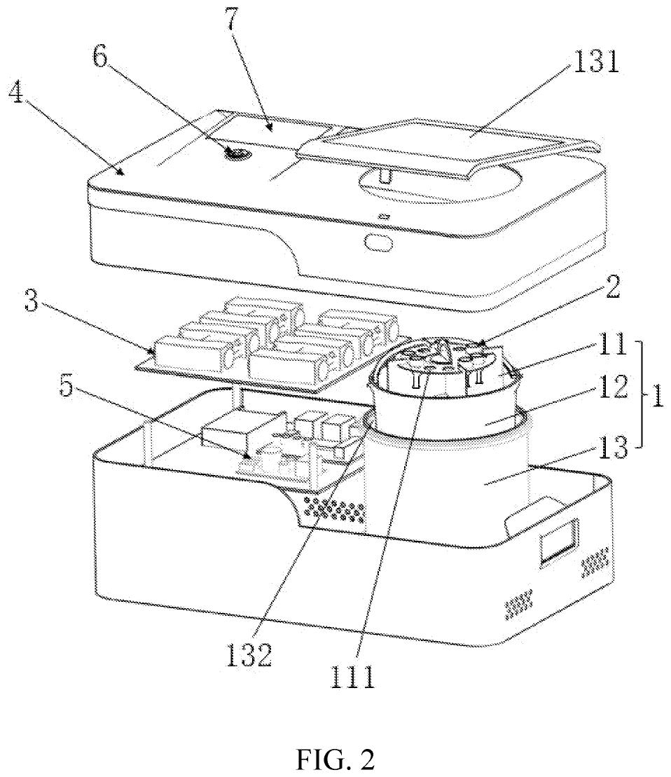

As shown in FIG. 2, the first embodiment of the present disclosure provides a negative-pressure drug loading apparatus for loading drug into the unloaded particles that are located in the particle storage tube 2. The negative-pressure drug loading apparatus includes a body 1 and a cover 131, and the body 1 is connected with the vacuum pumps 3 to realize the drug loading operation. The cover 131 can be opened and closed relative to the body 1, allowing for the removal and placement of the particle storage tube 2.

In its closed state, the cover 131 encloses the body 1 to form a sealed space. Within this space, at least one mounting slot 111 is included for securing the particle storage tube 2. When connected to the body 1, the vacuum pump 3 creates a negative-pressure within the mounting slot 111. It should be noted that the vacuum pump 3 can be replaced with other devices capable of generating negative-pressure.

The body 1 of this embodiment includes a mounting bracket 11, an inner sleeve 12 and an outer sleeve 13. Specifically, the mounting bracket 11 has a plurality of mounting slots 111. Each mounting slots 111 holds a particle storage tube 2, so that the unloaded particles 10 in the multiple particle storage tubes 2 can be loaded under negative-pressure at the same time, so as to improve the drug loading efficiency. The mounting bracket 11 can be either integrated with the inner sleeve 12 as a single unit or designed as separate pieces that can be assembled as needed. In this embodiment, each mounting slot 111 has an inner diameter between the first inner diameter and the second inner diameter, so that the housing portion 23 can pass through the mounting slot 111 but the guide portion 21 cannot pass through, thereby securing the guide portion 21 on the mounting bracket 11.

The mounting bracket 11 is placed in the inner sleeve 12, which is placed in the outer sleeve 13 to form sealed space after closing the cover 131. The particle storage tubes 2 are individually accommodated in the mounting slots 111. Therefore, the particle storage tube 2 can be taken and placed as a whole by directly lifting the inner sleeve 12, so as to improve the convenience and safety of taking and placing the particle storage tube 2.

Alternatively, the openable cover 131, arranged on the outer sleeve 13, can be inserted into the groove 132 between the outer sleeve 13 and the inner sleeve 12, thereby forming a sealed space within the outer sleeve 13. Furthermore, the outer sleeve 13 is designed to connect with the vacuum pump 3 to either pressurize or depressurize the sealed space. When both the mounting bracket 11 and the inner sleeve 12 are placed within the sealed space, the vacuum pump 3 can induce negative-pressure (below ambient pressure) in the space. Consequently, the unloaded particles 10 within the particle storage tube 2 can be loaded under this negative-pressure.

When the drug loading process is complete, the cover 131 can be opened to access the inner sleeve 12, which can then be removed from the outer sleeve 13 as a single unit. Subsequently, the particle storage tube 2 can be extracted from the inner sleeve 12 to retrieve the drug-loaded particles for treatment. For instance, in a scenario where the loaded drug solution is radioactive, these drug-loaded particles can be implanted into the body as radioactive seeds. Alternatively, if the drug-loaded particles are sufficiently small, they can be deployed into blood vessels or other human tissues through interventional surgical procedures.

It should be understood that the specific structure of the device, as described, represents just one embodiment and the present disclosure is not confined to this particular structural type. In an alternative embodiment, the body 1 might consist solely of an outer sleeve 13, with the inner sleeve 12 and the mounting bracket 11 integrated into it.

In the above embodiment, multiple vacuum pumps 3 are utilized, each operating independently and connected to the body 1. For specific applications, the number of vacuum pumps 3 operating simultaneously can be selected according to the requirements. It is understandable that a greater number of vacuum pumps 3 operating concurrently will result in a faster negative-pressure generation rate. Conversely, a smaller number will result in a slower rate. Moreover, because each vacuum pump 3 operates independently, the operation of one will not interfere with the others. Should one vacuum pump 3 fail, the normal operation of the remaining pumps will not be impacted.

As shown in FIG. 2, the negative-pressure drug loading apparatus in this embodiment features a portable design, enabling doctors to prepare the drug solution and load it into the unloaded particles minutes or hours before the operation. Thus, the drug solution and the drug loaded particles can be tailored to the individual needs of the current surgical patients for subsequent interventional or implantation surgery. Here, ‘portable design’ refers to meeting the following criteria: 1) the device's volume and weight are sufficiently limited to be moved by one person using only two hands; 2) it does not require external equipment such as a vacuum pump and can operate when connected to a power source. This portable design enables doctors to conduct the drug loading operation either within or in close proximity to the operating room, thereby minimizing radiation exposure from radioactive particles and also allowing for the immediate loading of drugs that cannot be stored for extended periods.

In summary, the negative-pressure drug loading apparatus provided by the first embodiment of the disclosure achieves the loading of drug liquid into the unloaded particles 10 within the particle storage tube 2 by applying negative-pressure. This method overcomes the issue of insufficient drug loading caused by the surface tension of the gas within the unloaded particles, thereby enhancing the efficiency and convenience of drug liquid loading.

Experiments have demonstrated that the negative-pressure drug loading method offered by this embodiment can achieve a drug loading capacity of 90% to 95% (of the cavity within the drug-loaded particles) for the drug-loaded particles. Additionally, the use of particle storage tubes 2 with different specifications allows the device to accommodate various types of drug-loaded particles available on the market, broadening its applicability. Ideally, the unloaded particles 10 could be those disclosed in the applicant's previous application, CN112190751A, or as disclosed in the applicant's previous application, CN111437265A.

### Second Embodiment

Building upon the first embodiment, as depicted in FIG. 2, the negative-pressure drug loading apparatus further comprises a housing 4 and a control unit 5. It will be apparent to those skilled in the art that the control unit 5 can be installed either inside or outside the device. It may be rigidly connected to the negative-pressure drug loading apparatus or designed to be detachable. In this embodiment, the approach of rigidly connecting the control unit inside the device is exemplified.

The housing 4 in this embodiment is designed as a single structure. It features an interior cavity that is specifically designed to house all the necessary components, including the body 1, the particle storage tube 2, and the vacuum pump 3. However, there is an alternative design option where the housing could be a split-type, which would only accommodate select components such as the vacuum pump 3. In this scenario, the body 1 would create a sealed space outside the housing 4.

The body 1 is centrally positioned within the housing 4. Protruding from the housing 4 is the top of the outer sleeve 13. The cover 131 is attached to this outer sleeve and can be manipulated to open or close, thus sealing or revealing the outer sleeve. Inside the housing 4, the control unit 5 is situated and is electrically linked to the vacuum pump 3. This connection allows the control unit 5 to manage the pump's operation, controlling its pressurization or pepressurization as required.

In the above embodiment, the negative-pressure drug loading apparatus further includes a control switch 6 and a display unit 7. The control switch 6 is connected with the control unit 5 to control the start or stop of the vacuum pump 3. The display unit 7 is connected with the control unit 5 for display. The specific display content can be the pressure change in the body 1, or the negative-pressure operation time, standing time, etc. In this embodiment, the display unit 7 is a liquid crystal display screen, but is not limited to this structure type.

### Third Embodiment

The difference from the first or second embodiment is that this embodiment is a portable design with an external vacuum pump.

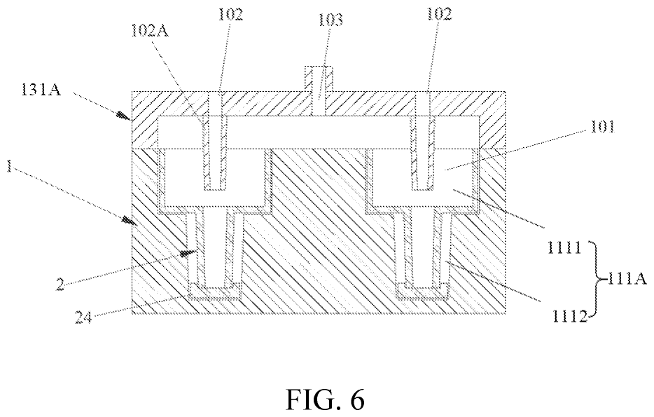

In this embodiment, the body 1 is an integral design, which is roughly in the shape of a column. The longitudinal section view of the body 1 is shown in FIG. 6. A closed space 101 is formed inside the body 1. In addition, there are a plurality of mounting slots 111A in the body 1 (take two in the figure as an example). The closed space 101 in the body 1 may be composed of a plurality of mounting slots 111A; It may also include a plurality of mounting slots 111A and other spaces (for example, the space above the mounting slot 111A in FIG. 6). Preferably, the closed space 101 is only composed of a plurality of mounting slots 111A (in other words, when the cover 131A is closed on the body 1, there is only a space formed by the mounting slot 111A inside the cover 131A and the body 1, that is, there is no other space mentioned above). During operation, open the cover 131A and place the empty particle storage tubes 2 directly in the mounting slots 111A to position the particle storage tube 2.

In this embodiment, the shape of the mounting slot 111A matches the external contour of the particle storage tube 2 to accommodate and position the particle storage tube 2 without hindering the removal of the particle storage tube. In this embodiment, the empty particle storage tube 2 is put into the mounting slot 111A in two cases:
1) the empty particle storage tube 2 has no cap 24, and the corresponding cap 24 is pre-set at the bottom of the mounting slot 111A; the empty particle storage tube 2 is inserted into the mounting slot 111A, and enable the second opening 231 to be accurately inserted into the cap 24; and the cap 24 can be easily taken out when the particle storage tube 2 is taken out; or
2) The empty particle storage tube 2 each has a cap 24. Put the empty particle storage tube 2 together with the cap 24 into the mounting slot 111A at the same time.

Therefore, the mounting slot 111A includes a guide cavity 1111 and an accommodation cavity 1112 for accommodating the guide portion 21 and the housing portion 23, respectively. Corresponding to the contour of the particle storage tube 2, the guide cavity 1111 is located above the accommodation cavity 1112, and connected each other. The inner diameter of the guide cavity 1111 is slightly larger than the outer diameter of the particle storage tube 2 (slightly larger by about 1-2 mm), and is larger than the inner diameter of the accommodation cavity 1112. The inner diameter of the accommodation cavity 1112 is larger than the outer diameter of the cap 24. Preferably, the inner diameter of the accommodation cavity 1112 is 0.6 mm to 1.2 mm to adapt to different specifications of the particle storage tube 2.

The cover 131A is mounted atop the body 1 and is designed to be operable, allowing it to be opened and closed as needed. The cover 131A has an inlet 102 and a connection part 103. The number of inlets 102 corresponds directly to the number of mounting slots 111A, with each inlet 102 positioned directly above its respective mounting slot 111A. It is ideal for the center-lines of the inlets 102, mounting slots 111A, and the housing portion 23 of the particle storage tube 2 to be aligned when the particle storage tube is secured in the mounting slots.

The inlet 102 is connected with the closed space 101 (that is, the inlet 102 passes through the cover 131A) and is used to fill the drug solution or particles into the particle storage tube 2 fixed in the mounting slot 111a. It can be understood that the filling port 102 can be used to fill the liquid medicine or particles manually or automatically.

The connection part 103 interfaces with the closed space 101 and is configured to connect with the vacuum pump 3, so that the closed space 101 can be pressurized or evacuated by the vacuum pump 3.

More preferably, the cover 131A further includes an extension tube 102A, designed to ensure that liquid medicine or particles entering from the inlet 102 accurately fall into the housing portion 23 (provided that the particle storage tube 2 is installed in the mounting slot 111A). The extension tube 102A is coaxially aligned with the inlet 102 and the accommodation cavity 1112, and it has a gradually decreasing inner diameter from top to bottom (with the minimum inner diameter being greater than the outer diameter of the particles). This design allows the extension tube 102A to guide particles or liquid medicine from the inlet 102 to a position closer to the accommodation cavity 1112.

The negative-pressure drug loading apparatus described in this embodiment is suitable for both portable use and fixed installations. In portable applications, it facilitates doctors to manually create particle storage tubes of various specifications (only a few tubes of the same specification can be manufactured in a single operation). Alternatively, the device can be integrated into an automated production line, enabling the batch production of particle storage tubes with identical specifications, where the particles within the particle storage tubes maintain the same drug loading composition.

It is understood that inlet 102 and connection part 103 can be combined into a common port. That is, first inject the liquid medicine with the common port, and then connect the common port with the vacuum pump for negative-pressure operation.

### Fourth Embodiment

In the third embodiment, the procedure involves placing empty particle storage tubes into the mounting slots, then closing the cover to initiate the process of loading the unloaded particle 10 into the empty particle storage tubes, followed by conducting the drug loading operation into the unloaded particle 10. Specifically in the present embodiment, once the empty particle storage tubes have been filled with the unloaded particles 10 are positioned in the mounting slots, the cover body is secured to load the drug solution into the unloaded particle 10. This embodiment is particularly well-suited for operations on a production line.

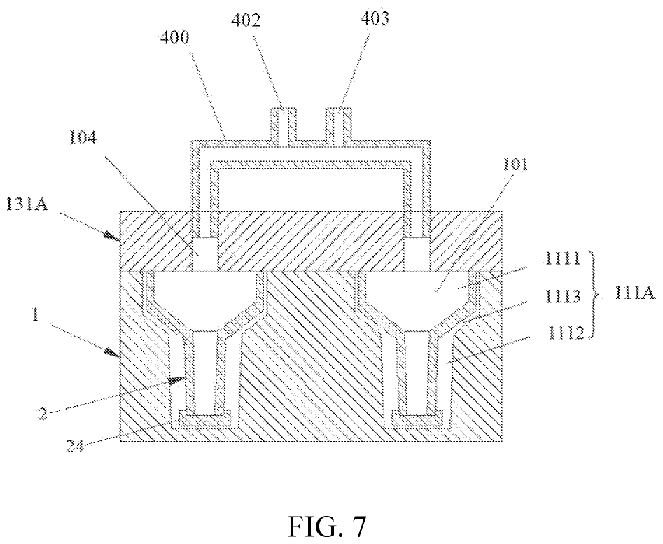

Specifically, as shown in FIG. 7, the body 1 forms a plurality of closed spaces 101, each of which has a mounting slot 111A for placing a particle storage tube 2. The mounting slot 111A includes a guide cavity 1111, an accommodation cavity 1112, and a transition cavity 1113 therebetween. The inner diameter of the guide cavity 1111 is slightly larger than the outer diameter of the particle storage tube 2 (being larger by about 1-2 mm), and is larger than the inner diameter of the accommodation cavity 1112. The inner diameter of the accommodation cavity 1112 is larger than the outer diameter of the cap 24. For example, the inner diameter of the guide cavity 1111 is 5-10 mm, and the inner diameter of the accommodation cavity 1112 is 0.5-1.2 mm, which is used to accommodate the particle storage tube. The particle storage tube has a guide portion 21 with an outer diameter of 4 mm and an accommodation portion 23 with an outer diameter of 0.9 mm.

In this embodiment, the cover 131B is similar to the cover 131A in the third embodiment. But, the cover 131B has only a plurality of inlets 102B. The number of the inlets 102B is the same as that of the mounting slots, and the inlets 102B is coaxial with the accommodation cavity, penetrating the closed space 101 for the external pipes 400.

When necessary, the inlet 102B is inserted with the pipeline 400 and then interface with a liquid port 402 and a gas port 403 set on the pipeline 100. Through the liquid port 402, the drug solution can be injected into the particle storage tubes 2 placed in the mounting slots manually or automatically. Finally, the gas port 403 is connected to the vacuum pump to pressurize or depressurize.

### Fifth Embodiment

This embodiment further introduces the specific steps of a negative-pressure drug loading method, detailing how to load the drug solution into the unloaded particles within the particle storage tube.

In the present example, the unloaded particle 10 has an outer diameter of 0.8 mm and a length of 10 mm, which is a common specification for radioactive particles. The micro-holes in the unloaded particles 10 have a diameter of 0.05 mm. Correspondingly, the inner diameter of the housing portion of the particle storage tube 2 is 1 mm, and its length is 52 mm, designed to accommodate 5 unloaded particles.

By using the negative-pressure drug loading apparatus shown in FIG. 2, the negative-pressure drug loading method provided by this embodiment specifically includes the following steps.

S51: Place the unloaded particles 10 into the housing portion 23 of each particle storage tube 2, with the second opening of the particle storage tube 2 being sealed by the cap 24.

S52: Inject a predetermined amount of drug solution into each particle storage tube 2, as depicted in FIG. 1, until the drug solution completely covers the unloaded particles 10.

The dosage of drug solution is determined according to the number and specification of unloaded particles in the particle storage tube. The inner diameter of the housing portion is also an influencing factor. In brief, the dosage of drug solution is 1.5 to 3 times of the sum of the volumes of all the inner cavities of the unloaded particles 10 in the particle storage tube 2, preferably 2 to 2.8 times. Taking the unloaded particles with a diameter of 0.8 mm and a length of 10 mm as an example, if there are 5 unloaded particles in the particle storage tube 2, and the housing portion has a diameter of 1 mm and a length of 12 mm, the injected drug solution is about 40 μL.

S53: Place all the particle storage tubes 2 into the mounting slots and close the cover 131 to seal the closed space.

S54: Depressurize the closed space through the vacuum pump 3 to form a negative-pressure in the unloaded particles 10 until no more bubbles appears in each particle storage tube 2.

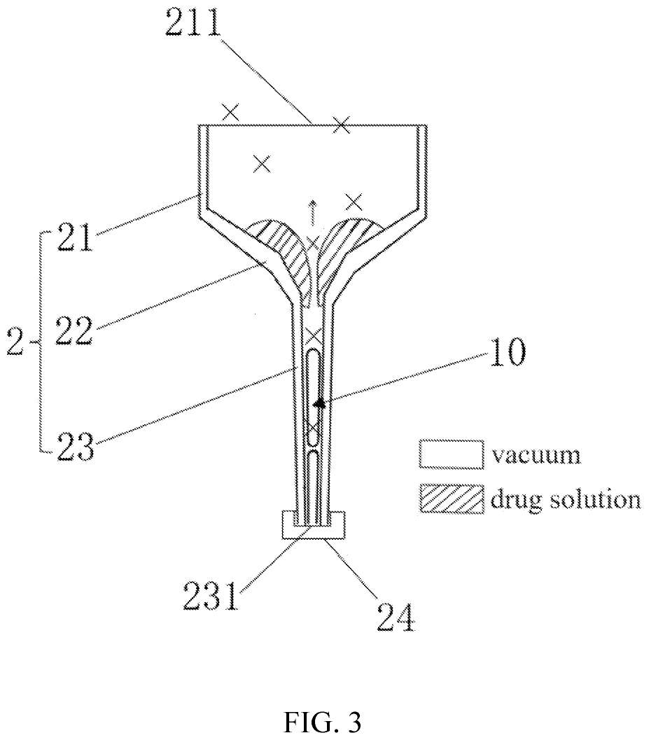

The vacuum pump 3 is used to provide negative-pressure within the body 1, so that the air can release from the liquid within the housing portion 23 and be extracted from the first opening 211 of the particle storage tube 2 until no more bubbles appear in the particle storage tube 2 (as shown in FIG. 3).

In this embodiment, the vacuum pump is set to a vacuum degree of 98 kPa and a pumping speed of 60 L/min. The specific values for pressure and pumping speed are determined based on the weight and size of the unloaded particles, as well as the diameter of the micro-holes, which can be determined through experimentation. It is important to note that the pressure and pumping speed must be pre-set according to the volume of drug solution, the volume within the housing portion, and the diameter of the micro-holes of the unloaded particles. This ensures a balance where the pressure is high enough to allow the air inside the unloaded particles to be quickly discharged (since the air pressure is greater than the weight of the liquid), but not so high that the drug solution is sucked out. In essence, the greater the volume of drug solution introduced into each particle storage tube, the greater the required negative-pressure. Additionally, the larger the volume of the housing portion, the longer the negative-pressure applied. Furthermore, the smaller the micro-holes of the particles, the greater the negative-pressure is required. Ultimately, the goal is to bring the inner cavity of the unloaded particles ideally to a vacuum degree of 98 kPa.

During the negative-pressure operation, a negative-pressure is applied to the entire closed space, resulting in the following effects: a negative-pressure is created on the surface of the drug solution. The gas inside the unloaded particles (under normal ambient pressure), which are immersed in the drug solution, is influenced by the pressure difference. This pressure difference causes the gas to escape from the micro-holes and move towards the first opening under the action of negative-pressure, facilitating the discharge of gas from within the unloaded particles.

S55: Stop the vacuum pump so that the pressure in the closed space gradually becomes greater than or equal to ambient pressure.

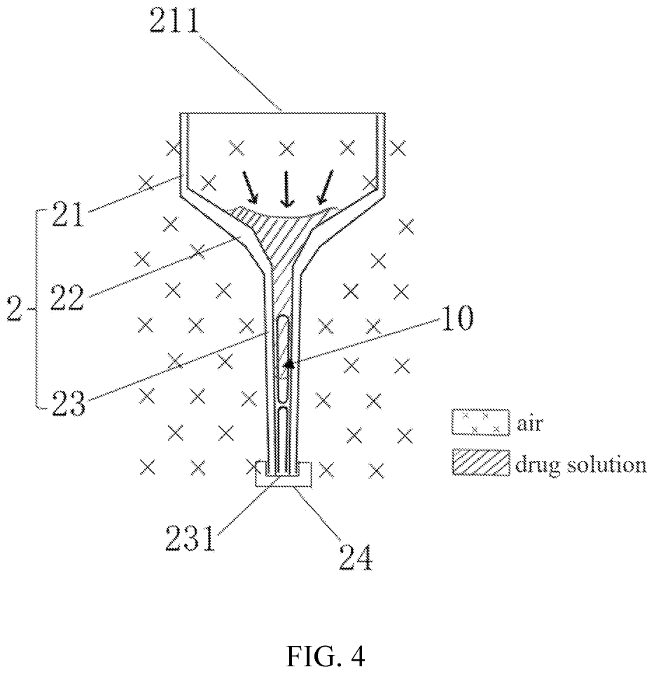

After the vacuum pumping is stopped, the pressure in the closed space can be restored to ambient pressure by standing for tens of seconds, so that the drug solution can soak into the housing portion 23 under the pressure and enter the interior of the unloaded particle 10 (as shown in FIG. 4).

This process is so slow that the drug solution can fully enter the inner cavity of the unloaded particles, thereby improving the drug loading ratio.

Preferably, the closed space can be pressurized to slightly greater than ambient pressure, increasing the pressure difference between the inside and outside of the unloaded particles, which is more conducive to improving the drug loading ratio. The greater the pressure difference is, the more drug solution will penetrate the interior of the unloaded particles until the entire inner cavity of the unloaded particles is saturated.

S56: after the pressure is stabilized to ambient pressure, open the cover and take out the particle storage tube.

The cover is finally opened to take out the particle storage tube for subsequent medical sterilization, disinfection, packaging and other operations.

### Sixth Embodiment

The present example further introduces a negative-pressure drug loading method for loading the unloaded particles in the particle storage tube without the need to pre-add the drug solution. Here, the negative-pressure drug loading apparatus shown in FIG. 6 is taken as an example, but the negative-pressure drug loading apparatus shown in FIG. 7 can also be used. In this embodiment, the negative-pressure drug loading method comprises the following steps.

S61: Place the set number of unloaded particles 10 into the particle storage tube 2.

With the second opening of the particle storage tube 2 being sealed by a cap 24, one or more unloaded particles 10 are placed into the particle storage tube 2.

S62: Place the particle storage tubes 2 in the mounting slots one by one, and close the cover to form a closed space.

The particle storage tube 2 containing the unloaded particles 10 in the mounting slots, is sealed within the closed space by the cover 131.

S63: Fill the predetermined amount of drug solution into the particle storage tube 2 through the filling port.

The specific amount of drug solution to be filled varies according to the number of the unloaded particles 10. The more the number of the unloaded particles 10, the greater the amount of the drug solution to be filled.

S64: Depressurize the closed space through the vacuum pump 3 to form a negative pressure in the inner cavity of the unloaded particle 10 until no more bubbles appear in the particle storage tube.

The inlet 102 is closed so that particle storage tubes 2 are sealed within the closed space. Then the vacuum pump 3 is connected to the connection part 103, providing negative pressure in the closed space in the body 1 through the vacuum pump 3, so as to extract all the air inside the unloaded particle 10. This extraction process is continued until the desired negative pressure is achieved in the unloaded particles 10 and beneath the drug solution. This ensures that the air inside the unloaded particles is pumped out while the drug solution remains undisturbed.

S65: stop the vacuum pump so that the pressure in the closed space gradually becomes greater than or equal to ambient pressure.

The vacuum pump 3 is then stopped, and the pressure above the drug solution is adjusted to be greater than or equal to the ambient pressure, allowing the drug solution to slowly penetrate the unloaded particles due to the pressure difference.

Specifically, once the negative pressure is achieved, the vacuum pump 3 stops and the pressure within the body 1 restores to ambient pressure. This allows the drug solution to enter the interior of the unloaded particles under the resulting pressure difference.

S66: After the pressure is stabilized to ambient pressure, open the cover and take out the particle storage tubes.

In order to ensure that the drug solution fully enters the unloaded particles and stabilize the pressure within the set range (e.g. 1.1 kpa), the drug loaded particles are formed by standing for a set time. Then, open the cover, close the guide portion of the particle storage tube with a plug (not shown), and take out the particle storage tube for routine operations of medical instruments such as sterilization and packaging.

To ensure that the drug solution fully penetrates the unloaded particles, the pressure in the closed space is maintained within the specified range (e.g., 1.1 kPa), and the particle storage tubes are rested for a predetermined duration to form the drug-loaded particles. Subsequently, the cover is opened, the particle storage tubes are plugged and then removed for standard medical instrument procedures, including sterilization and packaging.

### Seventh Embodiment

This example further introduces a negative-pressure drug loading method on the particle storage tube (empty particle storage tube) that is not pre-loaded with unloaded particles or drug solution. Here, the negative-pressure drug loading apparatus shown in FIG. 6 is taken as an example to illustrate the method.

In this embodiment, the negative-pressure drug loading method comprises the following steps.

S71: Place the empty particle storage tubes one by one into the mounting slots and close the cover to form a closed space.

S72: Place a predetermined number of unloaded particles through the filling port into each empty particle storage tube.

Since each inlet is coaxial to a mounting slot below, unloaded particles from the inlet will directly fall into the housing portion of the particle storage tube. It is important to note that due to the small size of the housing portion and the unloaded particles, only one unloaded particle can be placed in the particle storage tube at a time. This prevents the unloaded particles from getting stuck between each other, which could impede their normal descent into the housing portion. Consequently, this design ensures that any multiple particles in the housing portion are arranged in a series.

S73: Fill the predetermined amount of drug solution into the particle storage tubes through the filling port.

S74: Through the connection port, the vacuum pump depressurizes the inner space of the unloaded particles, until there is no bubbles in the particle storage tubes.

S75: Stop the vacuum pump so that the pressure in the closed space gradually becomes equal to ambient pressure.

S76: After the pressure is stabilized to ambient pressure, open the cover and take out the particle storage tubes.

The steps S73 to S76 are similar to those described in steps S63 to S66 of the sixth embodiment and are therefore not repeated

During the implantation process, firstly the cap is removed from the particle storage tube 2. Then, the drug loaded particles are pushed into a puncture needle by a push rod so as to implant the drug loaded particles into tissues or blood vessels.

Thus, the negative-pressure drug loading method can effectively complete the drug solution loading to the unloaded particles 10. It improves the drug loading operation efficiency, reducing the time required for drug loading of multiple particles in the same batch to approximately 1 minute. More importantly, this method can increase the drug loading capacity of the unloaded particles to more than 90%, or even beyond 95%. The drug loading capacity refers to the ratio of the volume of the drug solution within the drug-loaded particles to their effective volume.

It should be noted that the technical features in the above embodiments can be combined as required. In order to simplify the description, all possible combinations of the technical features in the above embodiments are not described. However, as long as there is no contradiction in the combination of these technical features, they should be considered as the scope recorded in this specification.

Compared with the prior art, the negative-pressure drug loading apparatus and the method provided by the disclosure offer the following technical effects.
1) the negative-pressure drug loading method allows for the unloaded particles with micro-holes (less than 1 mm) to be loaded with drug solution, thereby improving the drug loading rate and ratio;
2) operations are simple and efficient, enhancing both the efficiency and convenience of drug loading operations. It typically takes only 1-2 minutes to load drugs at a time.
3) the device has a simple and compact structure, making it suitable for portal use as well as for large-scale production.
4) with its low cost and straightforward operation, it is well-suited for widespread adoption.

The negative-pressure drug loading apparatus and the negative-pressure drug loading method provided by the disclosure are described in detail above. For those skilled in the art, any obvious changes made to the disclosure without departing from the essence of the disclosure will constitute an infringement of the patent right of the disclosure and will bear corresponding legal liabilities.

1. A negative-pressure drug loading apparatus, for loading under negative pressure unloaded particles placed in a particle storage tube, comprising:
a body having at least one mounting slot for positioning the particle storage tube, and
a cover configured to cover the body to provide a closed space under negative pressure, and enable drug solution enters inner cavities of the unloaded particles through micro-holes opened on the unloaded particles.

2. The negative-pressure drug loading apparatus according to claim 1, wherein:
the mounting slot includes a guide cavity and an accommodation cavity,
the guide cavity is located above the accommodation cavity, and an inner diameter of the guide cavity is larger than that of the accommodation cavity.

3. The negative-pressure drug loading apparatus according to claim 2, wherein:
an inner diameter of the accommodation cavity is 0.6 mm˜1.2 mm to adapt to different specifications of the particle storage tubes.

4. The negative-pressure drug loading apparatus according to claim 3, wherein:
the cover is provided with a same number of inlets as the mounting slot, and the inlets are connected with the closed space for filling the drug solution into the particle storage tube or feeding the unloaded particles.

5. The negative-pressure drug loading apparatus according to claim 4, wherein:
the cover is also provided with a connection part for connecting with a vacuum pump, and the connection part is connected with the closed space.

6. The negative-pressure drug loading apparatus according to claim 1, wherein:
the body comprises a mounting bracket, an inner sleeve and an outer sleeve;
the mounting bracket is provided with a plurality of the mounting slots for installing the particle storage tubes;
the mounting bracket is placed in the inner sleeve;
the inner sleeve is placed in the outer sleeve;
the cover is configured to seal or reveal the outer sleeve, thereby forming the closed space;
the outer sleeve is suitable to connect with a vacuum pump.

7. The negative-pressure drug loading apparatus according to claim 6, further comprising a housing, a control unit and a vacuum pump;
wherein the body and the vacuum pump are both arranged in the housing, and the cover can be opened and closed on the housing to seal or open the outer sleeve;
the control part is arranged in the housing to electrically connect with the vacuum pump and control the vacuum pump to pressurize or depressurize the closed space.

8-10. (canceled)

11. A negative-pressure drug loading method, for being used in the negative-pressure drug loading apparatus according to claim 1.

12. The negative-pressure drug loading method according to claim 11, comprising the following steps:
placing a particle storage tube into a mounting slot of a body, and closing a cover to the body to form a closed space within the body;
placing a predetermined number of unloaded particles and a predetermined amount of drug solution into the particle storage tube through an inlet in the body;
depressurizing the closed space until no bubbles appear in the particle storage tube;
stopping the depressurization and resting until the pressure in the closed space turns to be greater than or equal to ambient pressure;
opening the cover and taking out the particle storage tube in which the unloaded particles have been loaded with the drug solution.

13. The negative-pressure drug loading method according to claim 12, wherein:
the predetermined amount of drug solution is determined according to the number and specification of the unloaded particles in the particle storage tube.

14. The negative-pressure drug loading method according to claim 13, wherein:
during the operation of depressurization, the pressure and pumping speed of the vacuum pump are pre-set according to the amount of the drug solution, the volume in the housing portion, and the micro-holes' diameter, to ensure a balance where the pressure is high enough to allow the air inside the unloaded particles to be discharged but not so high that the drug solution is sucked out.

15. The negative-pressure drug loading method according to claim 11, comprising the following steps:
placing a particle storage tube with a plurality of unloaded particles into a mounting slot of a body, and closing a cover to the body to form a closed space within the body;
injecting a predetermined amount of drug solution into the particle storage tube through an inlet in the body;
depressurizing the closed space until no bubbles appear in the particle storage tube;
stopping the depressurization and resting until the pressure in the closed space turns to be greater than or equal to ambient pressure;
opening the cover and taking out the particle storage tube in which the unloaded particles have been loaded with the drug solution.

16. The negative-pressure drug loading method according to claim 15, wherein:
the predetermined amount of drug solution is determined according to the number and specification of the unloaded particles in the particle storage tube.

17. The negative-pressure drug loading method according to claim 16, wherein:
during the operation of depressurization, the pressure and pumping speed of the vacuum pump are pre-set according to the amount of the drug solution, the volume in the housing portion, and the micro-holes' diameter, to ensure a balance where the pressure is high enough to allow the air inside the unloaded particles to be discharged but not so high that the drug solution is sucked out.

18. The negative-pressure drug loading method according to claim 11, comprising the following steps:
placing a plurality of unloaded particles in a particle storage tube, and then injecting a predetermined amount of drug solution into the particle storage tube;
placing the particle storage tube into a mounting slot of a body, and closing a cover to the body to form a closed space within the body;
depressurizing the closed space until no bubbles appear in the particle storage tube;
stopping the depressurization and resting until the pressure in the closed space turns to be greater than or equal to ambient pressure;
opening the cover and taking out the particle storage tube in which the unloaded particles have been loaded with the drug solution.

19. The negative-pressure drug loading method according to claim 18, wherein:
the predetermined amount of drug solution is determined according to the number and specification of the unloaded particles in the particle storage tube.

20. The negative-pressure drug loading method according to claim 19, wherein:
during the operation of depressurization, the pressure and pumping speed of the vacuum pump are pre-set according to the amount of the drug solution, the volume in the housing portion, and the micro-holes' diameter, to ensure a balance where the pressure is high enough to allow the air inside the unloaded particles to be discharged but not so high that the drug solution is sucked out.
